(19) United States
(12) Patent Application Publication
(10) Pub. No.: US 2015/0201882 A1
(43) Pub. Date: Jul. 23, 2015

(54) CONTINUOUS REAL-TIME CSF FLOW MONITOR AND METHOD

(71) Applicant: ShuntCheck, Inc., Bensalem, PA (US)

(72) Inventors: Marek Swoboda, Philadelphia, PA (US); Matias Gabriel Hochman, Philadelphia, PA (US)

(21) Appl. No.: 14/417,932

(22) PCT Filed: Jul. 25, 2013
(86) PCT No.: PCT/US2013/052018

§ 371 Date: Jan. 28, 2015

Related U.S. Application Data
(60) Provisional application No. 61/742,048, filed on Aug. 2, 2012.

(51) Int. Cl. A61B 5/00; A61M 27/00; A61B 5/01

(52) U.S. Cl. CPC A61B 5/4851; A61B 5/01; A61B 5/4058; A61M 27/006; A61B 5/4836; A61B 5/7278; A61B 2562/0271; A61B 2562/043; A61B 2560/0412; A61B 2562/164; A61B 2560/0223

(57) ABSTRACT

An apparatus and method for determining a continuous CSF flow rate in an implanted CSF shunt in real-time. The system/method utilize a Peltier sensor formed on a flexible pad that is placed against the patient's skin. The Peltier sensor includes a Peltier device coupled to a thermal resistor that is contact with the patient's skin over the CSF shunt location. The Peltier device is operated continuously, controlled by the Peltier temperature sensor to a predetermined temperature that is below the patient's core temperature to form a temperature differential that causes any heat generated by the skin/CSF flow to be detected by a skin temperature sensor and the Peltier temperature sensor. Upstream and downstream temperature sensors, as well as control temperature sensors, are utilized to form a zero flow rate baseline that is used to calibrate a Peltier signal that corresponds to a real-time CSF flow rate. A sensor processing device processes all sensor data for generating the zero flow rate baseline and the Peltier signal.

### CROSS-REFERENCE TO RELATED APPLICATIONS

This PCT application claims the benefit under 35 U.S.C. §119(e) of Provisional Application Ser. No. 61/742,048 filed on Aug. 2, 2012 entitled CSF FLOW MONITOR and whose entire disclosure is incorporated by reference herein.

### BACKGROUND OF THE INVENTION

1. Field of Invention

This present invention generally relates to cerebrospinal fluid (CSF) shunts and, more particular, to a device and method for a continuous, real-time (CRT) monitor of CSF flow through shunt tubing implanted under the skin in hydrocephalus patients.

2. Description of Related Art

Hydrocephalus is a condition in which CSF accumulates in the brain ventricles, potentially leading to brain damage and death. Approximately 69,000 people are diagnosed with hydrocephalus each year in the United States. Hydrocephalus affects 300,000 Americans and generates $2 billion in annual US healthcare costs.

Hydrocephalus is corrected by placing a ventricular-peritoneal (VP) shunt that drains excess CSF to the abdomen (FIG. 1). However, shunts fail, typically by obstruction. Since catheter replacement requires surgery, a need for shunt revision must be established.

Additionally, regular, ongoing clinical management of shunted hydrocephalus patients is also complex. CSF over drainage can result in symptoms. In addition, clinical information about the performance of specific shunt valves and siphon control devices is limited. And adjustment of programmable shunt valves is a long, iterative process.

Current methods for detecting shunt malfunction and for optimizing shunt function do not meet the needs of hydrocephalus patients. Physical examination, including pumping of the shunt reservoir, is unreliable. Computed tomography (CT) scans remain the gold standard, but are expensive and cannot be used to investigate every headache, and results in repeated radiological exposures of patients (often children). Radionuclide shunt flow testing is invasive and poses a risk of infection. New technologies under development are complex (advanced magnetic imaging resonance (MRI) techniques, ultrasound tracking of bubbles), lacking in precision (forward looking infrared, FLIR) or require implantation (implanted thermal flow technologies) and have not reached the clinic.

The need for new diagnostic tools for hydrocephalus patients is highlighted by the NH announcement “Advanced Tools and Technologies for Cerebrospinal Fluid Shunts” (PA-12-190).

U.S. Patent Application No. 2013/0109998 (which is incorporated by reference in its entirety herein), owned by the same Applicant as the present application, namely, ShuntCheck, Inc., discloses a non-invasive device for determining CSF flow rate through shunts and is hereinafter referred to as “ShuntCheck”. ShuntCheck is a system that utilizes an array of thermo-sensors clustered in three sections that applies a finite cooling source (e.g., an ice cube) in close proximity to the shunt and to the thermo-sensors. The thermo-sensors are coupled to an analyzer to that utilizes the thermo-sensor data to calculate the CSF flow rate. In particular, as shown in FIGS. 2A-2D, a ShuntCheck thermosensor (FIG. 2A) forms a skin thermometer that is placed over a shunt catheter where it crosses the patient's clavicle; the shunt is then chilled upstream via an instant ice pack. As shown in FIG. 2B, a micro-pumper device (see U.S. Patent Publication No. 2013/0102951, also owned by ShuntCheck, Inc. and which is also incorporated by reference in its entirety herein) vibrates the shunt valve, generating a temporary increase in shunt flow in patent, but not in occluded shunts, thereby enabling the ShuntCheck to differentiate intermittently flowing patent shunts from obstructed shunts. As shown in FIG. 2C, the ShuntCheck sensor comprises a middle sensor positioned directly over the shunt plus two controls sensors which read ambient skin temperature. As shown in FIG. 2D, if cooled CSF reaches the test sensor, CSF flow is indicated; ShuntCheck's computer screen (e.g., a tablet computer) reports the result as “Flow Confirmed” or “Flow NOT Confirmed. It should be noted that a temperature drop of greater than or equal to 0.2° C. indicates normal flow (greater than 5 ml/hr).

However, the short duration of the test limits its utility for shunt valve adjustment, investigating suspected shunt over-drainage, etc.

In contrast, a non-invasive, non-radiologic device which can track changes in CSF flow rate in real-time over extended time periods would address many ongoing clinical management needs and become a valuable tool for the neurosurgery clinic. Thus, there remains a need for a device and method capable of a continuous, real-time (CRT) monitor of CSF flow through shunt tubing implanted under the skin in hydrocephalus patients

All references cited herein are incorporated herein by reference in their entireties.

### BRIEF SUMMARY OF THE INVENTION

An apparatus for determining cerebrospinal fluid (CSF) flow rate in an implanted CSF shunt in real-time is disclosed. The apparatus comprises: a pad that is placed against the skin of a patient over the location of the CSF shunt, and wherein the pad itself comprises: a Peltier sensor comprising: a Peltier device that is operated continuously and which is displaced away from a first surface of the pad via a thermal resistor over the location of the shunt; and a first set of temperature sensors (e.g., thermistors) associated with the Peltier device for detecting heat generated from the patient's skin and any CSF flow through the shunt; and a sensor processing device that is electrically coupled to the pad for receiving temperature data from said first set of temperature sensors, and wherein the temperature data from the first set of temperature sensors is used by the sensor processing device to determine a continuous real-time flow rate of the CSF through the CSF shunt.

A method for determining cerebrospinal fluid (CSF) flow rate in an implanted CSF shunt in real-time is disclosed. The method comprises: applying a Peltier device, via a thermal resistor, against the skin of a patient over the location of the CSF shunt; positioning a first temperature sensor (e.g., a thermistor) between the Peltier device and the thermal resistor and positioning a second temperature sensor between the thermal resistor and the skin of the patient; energizing the Peltier device on a continuous basis and using the first temperature sensor to control the Peltier device to maintain a predetermined temperature; detecting a temperature gradient between the first and second temperature sensors from temperature data generated by the first and second temperature sensors; and processing the temperature gradient to determine a Peltier signal that corresponds to a continuous real-time flow rate of the CSF through the CSF shunt.

An apparatus for determining cerebrospinal fluid (CSF) flow rate in an implanted CSF shunt in real-time is disclosed. The apparatus comprises: a pad that is placed against the skin of a patient over the location of the CSF shunt and wherein the pad comprises: a Peltier sensor that itself comprises: a Peltier device that is operated continuously with a first set of temperature sensors (e.g., thermistors) associated with the Peltier device for detecting heat generated from the patient's skin and any CSF flow over the location of the shunt; a second set of temperature sensors (e.g., thermistors) arranged upstream and downstream of the Peltier device for detecting a temperature distribution along a path of the CSF shunt; and a sensor processing device that is electrically coupled to the pad for receiving temperature data from the first set of temperature sensors and from the second set of temperature sensors, the temperature data from the second set of temperature sensors being used by the sensor processing device to define a zero flow baseline signal for calibrating the Peltier sensor and wherein the sensor processing device further uses the temperature data from the first set of temperature sensors in conjunction with the zero flow baseline signal to determine a continuous real-time flow rate of the CSF through the CSF shunt.

### BRIEF DESCRIPTION OF SEVERAL VIEWS OF THE DRAWINGS

The invention will be described in conjunction with the following drawings in which like reference numerals designate like elements and wherein:

FIG. 1 is a diagram showing how ventricular-peritoneal (VP) CSF shunt extends from an inflow catheter in a patient's brain ventricle to the abdomen;

FIG. 2A depicts a ShuntCheck thermosensor that is placed over the shunt catheter (not shown, beneath a patient's skin) where it crosses the clavicle;

FIG. 2B depicts a Micro-Pumper device that vibrates the shunt valve, generating a temporary increase in shunt flow in patent, but not in occluded shunts, thereby enabling the ShuntCheck to differentiate intermittently flowing patent shunts from obstructed shunts;

FIG. 2C depicts a ShuntCheck sensor that comprises a middle sensor directly over the shunt and including two controls which read ambient skin temperature;

FIG. 2D is a ShuntCheck computer screen that indicates whether there is or is no CSF flow; in this figure, CSF shunt flow is confirmed;

FIG. 3 is an isometric view of the components of the present invention;

FIG. 3A is a plot of the downstream sensor signal versus flow rates;

FIG. 4A is a side view functional diagram of the Peltier Sensor of the present invention;

FIG. 4B is a top view functional diagram of the Peltier Sensor of the present invention taken below the Peltier device;

FIG. 5 depicts a graph of the Peltier Sensor Signal vs. Flow rates for differentiating low vs. robust flow rates;

FIG. 6 depicts a graph of the ArcTangent³ of the T_{DN}/T_{UP} number, where T_{DN} is the temperature downstream and T_{UP} is temperature downstream; this function can be utilized as a good separator between no flow (values greater than 1) and flow conditions (values lower than 0);

FIG. 7 is a graph depicting the Combined CRT Signal which is the Peltier Signal divided by the Zero Flow Signal vs. Flow Rate;

FIG. 8A is a thermal need probe, outfitted with thermistors, used during prototype testing on a test animal;

FIG. 8B depicts skin thickness measurements being taken on the test animal for calculating skin conductivity using the “buried pipe model”;

FIG. 9 is a functional block diagram of bench model of the CRT ShuntCheck prototype;

FIG. 10 depicts the CRT ShuntCheck prototype coupled to the flank of a test animal;

FIG. 11A depicts a graph of test data directed animal CRT signal data by flow rate, showing uncalibrated signals with error bars;

FIG. 11B is related to FIG. 11A but with zero flow calibrated signals;

FIG. 12A is a graph depicting the response of the CRT ShuntCheck concept to a pulsing flow pattern;

FIG. 12B is a graph depicting the response of the CRT ShuntCheck concept to a rising and falling flow pattern; and

FIG. 13 is a graph depicting the CRT signal of the CRT ShuntCheck prototype vs. flow rate change.

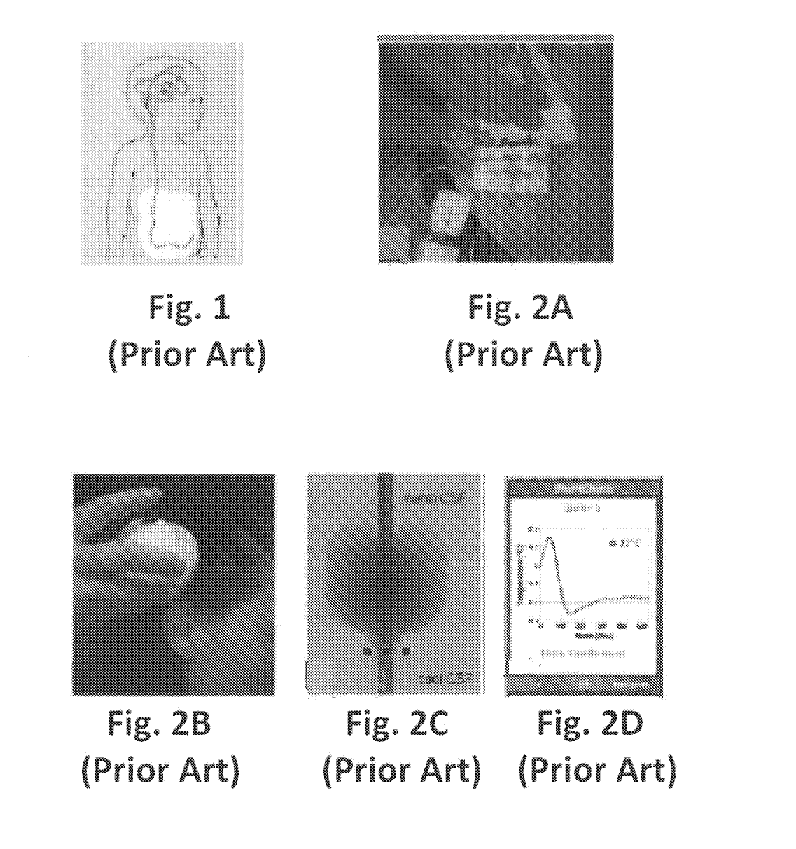

### DETAILED DESCRIPTION OF THE INVENTION

The present invention 500 is termed a “continuous real-time (CRT)” CSF flow monitor and method. This device provides improved care for hydrocephalus patients by providing a rapid and non-invasive method for monitoring changes in CSF flow in shunted patients. As a result, the present invention 500 directly responds to such demands for diagnostic tools for use in a hospital or outpatient settings that work in real-time to quantitatively determine shunt function.

As is discussed in detail later, a key aspect the present invention 500 uses non-invasive thermal dilution technology to monitor changes is CSF flow over extended periods of time, enabling neurosurgeons to assess in real-time the impact of changes—of valve setting, patient position, etc.,—on shunt flow. This cannot be accomplished in any way with current technologies and represents an important new tool for managing hydrocephalus. As will also be discussed later, a “Peltier sensor” of the present invention 500 represents a breakthrough which significantly increases the accuracy and utility of the continuous real-time flow monitor and method, also referred to as “continuous real-time (CRT) ShuntCheck.” The Peltier sensor is based on Peltier cooling and continuous thermal recordings which, when monitored, are indicative of changes in shunt flow.

The present invention 500 employs a breakthrough in thermal dilution technology which enables long term, continuous, real-time measurement of CSF shunt flow. As a result, the present invention 500 forms:
(1) a tool to streamline the process of adjusting shunt valve settings to accommodate individual needs for CSF drainage. While the settings for these valves in each patient must currently be determined empirically over a number of weeks, the invention 20 assists in measuring changes in CSF flow due to changes in the valve setting. (For example, the invention 20 can be used CRT to establish the initial valve setting—at the highest opening pressure which allows moderate CSF flow);
(2) a tool for assessing suspected over-drainage. CSF flow data allows neurosurgeons to identify periods and causes of high CSF flow when assessing suspected CSF over-drainage. This data can also be used to evaluate flow and siphon control devices to determine if they are meeting the patient's needs; and
(3) a post operative test to confirm shunt function. Hospitals in sparsely populated areas often conduct post-surgical CT scans to confirm shunt function before releasing patients for the long drive home. CSF flow data can confirm shunt function more quickly than CT (which requires time for the ventricles to stabilize).

More generally, the present invention can be used to establish a “normal” baseline flow pattern for each patient—similar to the current practice of establishing a normal baseline for ventricular size via imaging.

As shown most clearly in FIG. 3, the present invention 500 comprises a Peltier sensor 502 and a sensor processing device 504. The Peltier sensor 502, as will be discussed later, comprises a flexible base or patch 503 (also referred to as “pad”) that is placed in contact with the patient's skin 12 directly over the shunt 10 that is pre-disposed under the patient's skin 12. The arrows in the shunt 10 in FIG. 3 indicate the direction of CSF flow. The Peltier sensor 502 includes a housing 511 having a vent/grating 513 for an internal Peltier device 514 (e.g., CP60133 Peltier MOD 15×3.3 mm 6.0 A INP manufactured by CUI, Inc.) and comprises several temperature sensors or thermo-sensors (e.g., thermistors, 103JT-025 Thermistor NTC 10 kΩ manufactured by Semitec, by way of example only) that provide temperature data to a CSF analyzer 504 via conductors 507 (or can form a wireless connection to the sensor processing device 504). A wire harness 505 can include conductors 507 along with power conductors 509 from a power/controller device 515 used to energize/control the Peltier device 514 and a fan 520A for heat dissipation purposes, as will also be discussed later. The sensor processing device 504 collects the data from the thermo-sensors and includes a display 504A and keypad or other input mechanism 504B. The sensor processing device 504 includes all of the appropriate analog-to-digital (A/D) conversion circuitry/interfacing and associated microprocessor or microcontroller processing necessary to analyze the temperature data collected from all of the temperature sensors in accordance with the method of the CSF shunt flow analysis discussed below. The temperature data can be processed and outputted (e.g., via a the display 504A or other output means) directly from the sensor processing device 504, or the temperature data can be wirelessly transmitted from the sensor processing device 504 to a remote device (not shown) where the temperature data is analyzed and the results displayed at the remote device.

It is within the broadest aspect of this invention 500 to include a Peltier device and fan power source within the sensor processing device 504 and that the illustrated example does not, in any way, limit the sensor processing device 504 configuration. The use of the term “sensor processing device” 504 implies all aspects of powering and controlling the Peltier device 514 as its associated heat dissipating fan 520A, as well as any and all signal conditioning (A/D conversion, filtering, etc.) of all of the temperature sensor data/signals and the processing and analysis of this data into corresponding CSF flow status/rate outputs.

The Peltier sensor 502 adheres comfortably to the patient's skin (e.g., over the patient's clavicle) for extended periods of time, e.g., 30 minutes to two hours. An alternative is overnight use.

To permit the patient to be mobile during use of the present invention, the sensor connects to a belt-worn battery pack which connects wirelessly to the CSF analyzer 504. It should be understood that the sensor processing device 504 may analyze this temperature data directly or may transmit such data to another device for CSF flow analysis.

The device must track changes in CSF flow—differentiating no flow from low flow from robust flow. Differentiating low from robust flow is important for shunt valve adjustment and over-drainage assessment.

### Peltier Sensor 502

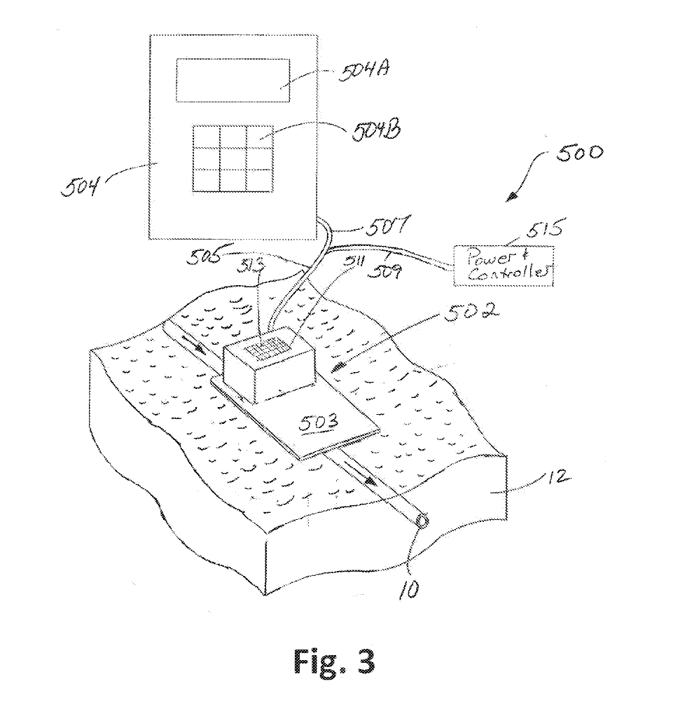

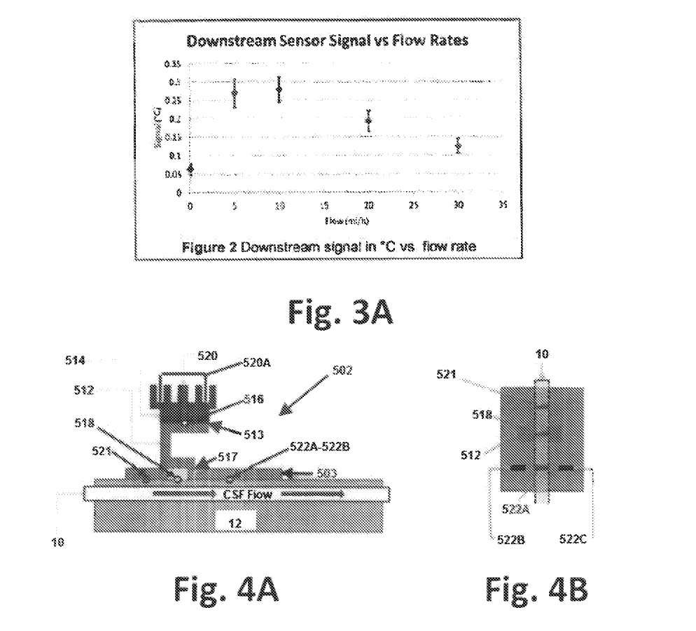

The Peltier sensor 502 of the present invention 500 is based upon the current ShuntCheck design, e.g., cooling upstream” (via a Peltier electronic cooling device) and thermal sensors “downstream” which detect changes in CSF flow as shown in FIG. 2C. However, as mentioned previously, such a configuration could track flow vs. no flow but could not differentiate low flow from robust.

As shown in FIG. 3A, low CSF flow of 5 ml/hr generates a stronger temperature signal than did robust flow rates of 20 ml/hr. It is from this observation that resulted in the development of the Peltier sensor 502—using the Peltier cooling device to track the heat transfer required to hold the skin above the shunt at a uniform temperature. (As flow of warm CSF increases, the Peltier must remove more heat to maintain a constant skin temperature).

As shown in FIGS. 4A-4B, the CRT thermal patch (“flexible patch”) 503 is placed over the location of the CSF shunt 10. The patch 503 utilizes three measurement modalities: upstream temperature, Peltier sensor and downstream temperature. FIG. 4A depicts the side-view of the Peltier sensor 502 (without the housing 511), with its patch 503 on the skin 12 and above the shunt 10. The Peltier device 514 is a flat heat pump which is warm on its top and cool on its bottom. A radiator 520 and fan 520A dissipate heat. The Peltier thermistor 516 controls the Peltier device 514. A thermal resistor 512 (e.g., a nylon/aluminum member) slows the transfer of heat from the skin to the Peltier device 514. The temperature difference between the skin thermistor 518 and the Peltier thermistor 516 is the “Peltier signal”. FIG. 4B depicts the top view of the patch portion of the Peltier sensor 502 and shows the alignment of the various patch thermistors over the shut catheter 10.

In particular, the Peltier sensor 502 comprises a thermal resistor 512 (e.g., an aluminum C-shaped element) secured (e.g., glued) to the Peltier device 514 using thermo conductive epoxy. A thermistor 516 is sandwiched between the Peltier device 514 and the thermal resistor 512 to control temperature at the cold end 513 of the resistor 512. A second thermistor 518 is attached to the other end 517 of the thermal resistor 512, the part which touches skin 12. An aluminum radiator 520 with a small 520A sits atop the Peltier device 514 to remove heat (the Peltier device 514 is cool on one side, warm on the other).

### Principle of Operation

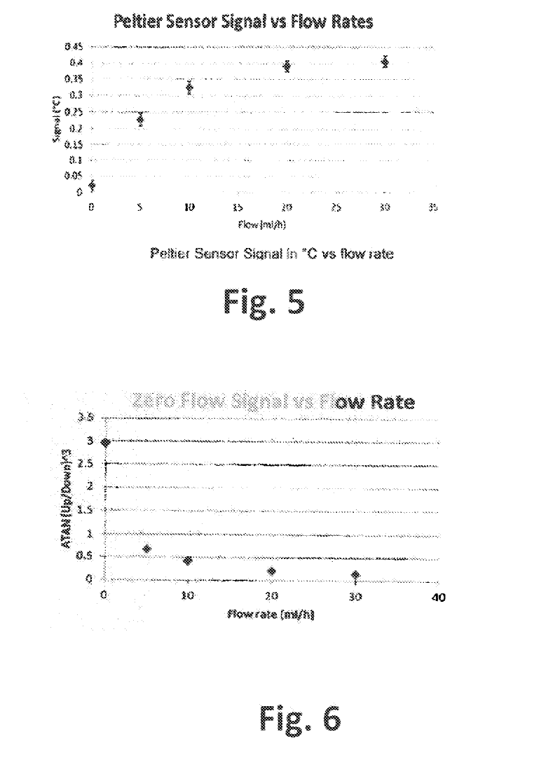

The bottom part of the thermal resistor 512 (e.g., 14×5 mm) cools down the tissue 12 surrounding the CSF shunt 10. This causes a temperature gradient between CSF inside the shunt tube 10 and the surrounding tissue 12. Due to the generated temperature gradient, the thermal energy from CSF flows to the tissue 12 and eventually, via the thermal resistor 512, to the Peltier device 514. The temperature difference between the Peltier thermistor 516 and skin thermistor 518 is proportional to the amount of heat flowing through the thermal resistor 512. Therefore, the amount of heat is in a direct relationship to the CSF flow rate and permits the differentiation of low vs. robust flow rates (FIG. 5).

While the Peltier sensor 502 has a monotonic characteristic in a wider range of flow rates (0 through 30 ml/h), it lacks a stable zero flow measurement. This is due to the fact that the tissue conductivity varies with perfusion, fat content, etc. In order to obtain stable zero two additional thermal measurements are provided. The first thermistor measures the temperature change over the shunt catheter 10 upstream of the Peltier device 514 and is positioned over the shunt 10 while the second thermistor measures temperature changes over the catheter 10 downstream of the Peltier device 514. In particular, an upstream thermistor 521 (FIGS. 4A-4B) is provided, along with downstream sensors 522A-522C. Thermistor 522A (also referred to as “test thermistor”) is positioned over the shunt 10, while thermistors 522B and 522C (also referred to as “control thermistors”) are positioned away from the shunt 10 to measure surrounding skin temperatures. It should be noted that although the control thermistors 522B/522C are shown aligned with the test thermistor 522A, this is not required; it is within the broadest scope of the invention to include these control thermistors 522B/522C in any remote location away from the Peltier device 514 and not aligned with the test thermistor 522A.

### Upstream Measurement (T_{UP})

The Peltier device 514 cools down the area of the skin in its near proximity. When there is no CSF flow, the upstream sensor 521 is cooled down by the Peltier device 514. When CSF starts flowing underneath the upstream sensor 521, it delivers heat. The temperature of the tissue under the upstream sensor 521 increases, an increase which is related to the CSF flow rate. Flows as low as 1-2 ml/h deliver enough heat to warm up the skin. The advantage of this method is its high sensitivity to flow. No flow conditions are easily detectable by a cold upstream sensor.

### Downstream Measurement (T_{DN})

The downstream measurement mimics the original ShuntCheck device (see FIGS. 2A-2D). In particular, a test thermistor 522A sits above and tracks the shunt catheter temperature and is flanked (or otherwise positioned in a non-aligned configuration) by two control sensors 522B and 522C which compensate for skin temperature variability. When CSF is not flowing, these thermistors 522A-522C register the same temperature. When CSF flows, the test thermistor (middle) 522A shows a drop in temperature relative to the control thermistors 522B/522C.

### Zero Flow Signal

The important advantage of the downstream and upstream sensors is their synergistic nature. Each of the sensors is an accurate “no-flow” detector, but the balance between the upstream and downstream sensor shows even greater accuracy due to the fact that during the test, the skin temperature may fluctuate. A single up or downstream measurement would be sensitive to such changes but the ratio of those signals is insensitive to body temperature. (FIG. 6).

The balance (ratio) between the upstream (T_{UP}) and downstream sensors (T_{DN}) indicates whether or not there is a flow in the shunt 10, thereby establishing an accurate zero-flow baseline regardless of the skin temperature. The vector defined by the temperature of the upstream thermistor 521 and the temperature of the downstream thermistor 522A, V_{up-down} (T_{UP}, T_{DN}) serves as a precise indicator of zero flow. Although the modulus of this vector is to a certain extent random, its phase is monotonically related to the flow rate with a precise zero value.

In particular, the graph of FIG. 6 depicts the ArcTangent³ of the T_{DN}/T_{UP} number. This function can be utilized as a good separator between no flow (values greater than 1) and flow conditions (values lower than 0).

### The Combined “CRT Signal”

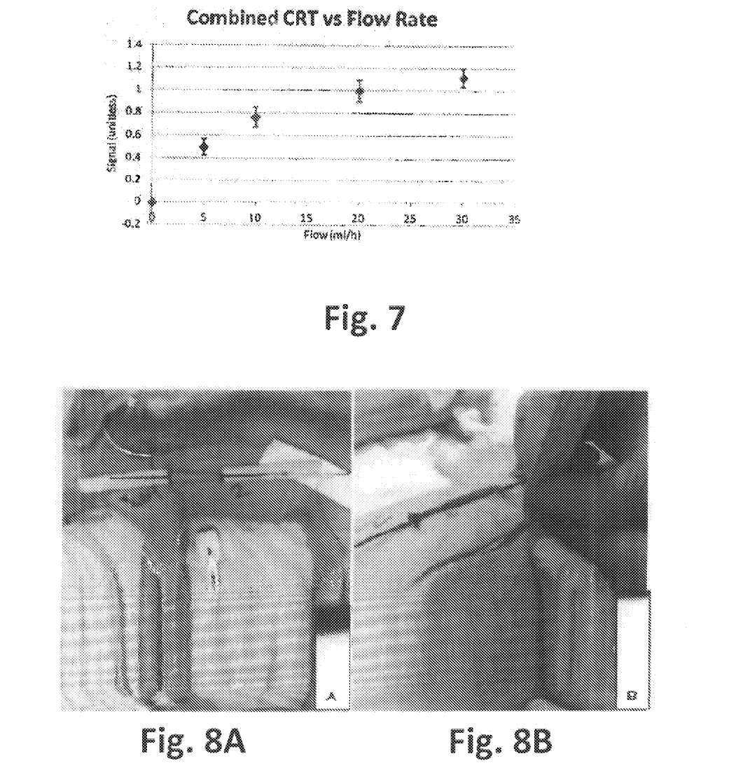

The combined signal takes advantage of the monotonic response of the Peltier sensor and zero-sensitive response of the Zero Flow Signal. FIG. 7 demonstrates that the Combined CRT Signal (the Peltier Signal/Zero Flow Signal) is monotonic within a wide range of flow rates and minimizes signal fluctuations around zero (due to division by the Zero Flow Signal which reaches high values at low flows). In particular, FIG. 7 is a graph depicting the Peltier Signal divided by the Zero Flow Signal vs. flow rate.

### Data Acquisition and Display System

The sensor processing device 504 was implemented using a data acquisition battery unit (“DAQ”) and a computer (e.g., a tablet computer). The DAQ powers and controls the Peltier device 514 and conditions and converts the analog temperature sensor signals into digital data. The computer receives the temperature sensor data and uses an application (e.g., LabView software) to display real-time results.

In view of the foregoing, the present invention 500 is able to generate smooth and non-overshooting cooling. As shown in FIG. 7, the present invention 500 is capable of measuring variable flow rates in the dynamic range of 0-20 ml/h.

### Prototype Equipment and Testing Results

A bench thermal skin simulator was implemented to match long term animal model thermal responses to cooling (using a pig model which closely matches human skin). Animal skin conductivity was assessed by using geometry resembling the geometry of the shunt system. A needle probe was constructed. A stainless steel needle (pipe) heater outfitted with temperature sensors was implanted under the skin (see FIGS. 8A-8B). The temperature difference between the pipe and the skin surfaces was measured. The heat transfer in such geometry is known as the “buried pipe model” and is governed by the equation:

$$q = k \frac{2 π}{ln ( \frac{2 D}{r} )} ( {T}_{pipe} - {T}_{skin} )$$

where q is heat exchange between the pipe and the surrounding material, k is heat conductivity, D is pipe depth, r is the pipe radius, Tₚᵢₚₑ is temperature of the pipe surface and Tₛₖᵢₙ is the temperature of the skin surface over the pipe. Since the geometry is known, q can be calculated from current and electrical resistance of the probe, temperatures are measured by two thermistors (one on the needle and another one on the skin surface), the equation can be solved for k.

The testing has been performed on the thermal skin simulator using 3 kg piglets which are FDA approved standard for the pediatric skin simulations. Both systems (the silicon skin simulator and the piglet skin) have been tested in order to measure thermal conductivity.

In particular, FIG. 8A depicts the thermal needle probe being inserted under the skin. The probe was outfitted with thermistors controlling its surface temperature as well as electric ports (on both ends) to deliver electrical current to heat up the device. The skin temperature thermistor was subsequently placed on the skin surface to measure thermal effects caused by the needle heater. After thermal measurements were completed, the skin thickness was measured, as shown in FIG. 8B. The calculation for skin conductivity was performed using the “buried pipe model.”

The results showed that piglet skin conductivity is 0.395 W/K m (see Table 1 below), which is consistent with the values reported in the literature.

TABLE 1
Mock and Pig skin thermal conductivities.

| | Thermal conductivity k [W/Km] |
|---|---|
| Silicone rubber mock skin | 0.303 stdev = 0 009 |
| (bench model) | |
| Animal skin | 0.395 stdev = .099 |

### Thermal Bench Model

An artificial skin was formed to replicate natural skin thermal conductivity using silicone rubber (Freeman Mfg & Supply Co.). The slightly lower silicone rubber conductivity (which is believed to be caused by blood circulation which is absent in the bench system) was mathematically compensated for during validation testing.

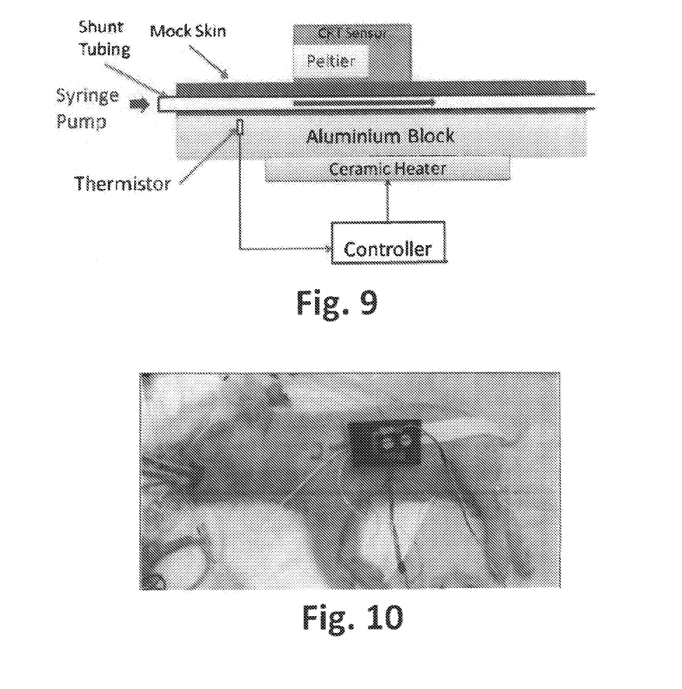

The bio-heat generated by human body was simulated by an active system controlled by a close-loop feedback. (The schematic of the bench is shown in FIG. 9). The system behaves like the natural skin trying to preserve constant skin temperature close to 34° C. Tissue heat capacitance was not emulated since the experiment was designed to reflect steady state phenomena (the measurement was performed after all temperatures in the system reached steady state. Time to reach steady state was approximately 5 minutes). Efforts to measure skin conductivity, including skin perfusion testing, were not taken since pertinent literature indicated that there is no consistent way of translating skin perfusion into skin conductivity. Additionally, researchers failed to observe measurable changes in conductivity due to perfusion. As a result, the “buried pipe model” was preferred since the geometry of the shunt system is perfectly represented in this model; the buried pipe model is suitable for steady state system and due to the Peltier cooling device, the present invention 500 can be considered steady state.

A thermal bench related to ShuntCheck thermal testing was used to validate the prototype's ability to monitor shunt flow rate over time. As shown in FIG. 10, a continuous, real-time sensor was placed on a flank of the pig. The Peltier device was cooled by a radiator and a battery of miniature fans. The shunt tubing was implanted under the skin of the leg and passed under the pig's flank. This configuration permitted the CSF fluid to equalize with the body temperature before it interacted with the sensor.

A randomize table of flow rates (0, 5, 10, 20, 30 ml/h) was used which covered a physiological range of CSF flows in shunt tubing. The bench and animal experiments were performed for approximately four hours, which resulted in 10 tests per flow rate.

Several levels of cooling temperatures were tested ranging from 6 through 14° C. below the baseline skin temperature (typical skin temperature 34° C.). The temperature of 14° C. was selected because it yielded the best signal to noise ratio while maintaining a very safe level of cooling.

### Test Results

### CRT Differentiates Zero, Low & Robust Flow

The CRT sensor tracks CSF flow rate changes over prolonged periods of time. The Combined CRT Signal presents a monotonic characteristic in animal and bench testing (with the bench corrected for heat conductivity). Four-hour tests were conducted in six animals. All tests showed that several hour monitoring is possible with the CRT sensor. The CRT system can provide a wide range of outputs for real time and offline analysis. The accuracy of the system can be enhanced with calibration to zero or with smoothing algorithms.

Real Time without Calibration

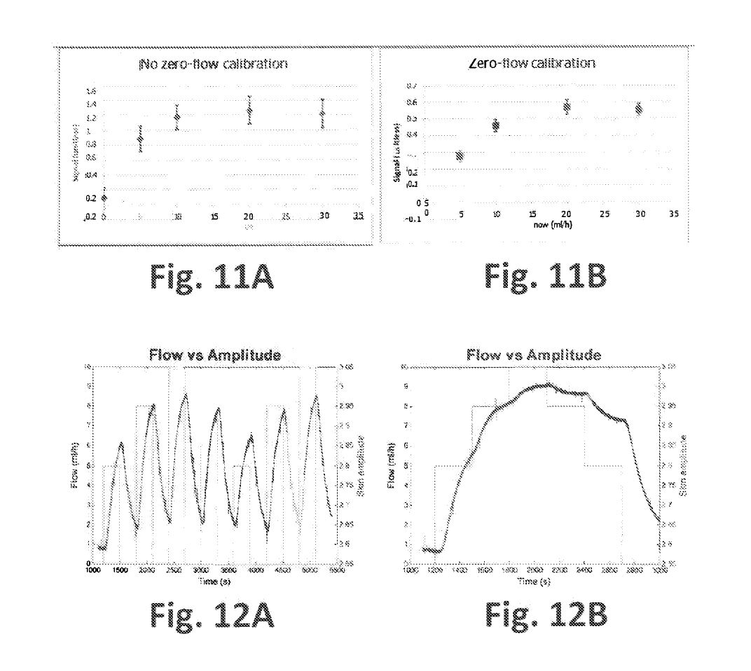

As shown in FIG. 11A, CRT can differentiate between no, low and robust flow, making it a useful tool for assessing changes in flow rates in an individual patient (e.g., for investigating suspected over-drainage). (Errors are 0.14 for 0 ml/h, and 0.19 for 30 ml/h. SN R=6.3. Additionally, CRT matches current ShuntCheck's ability to differentiate 10 ml/hr flow from 0 ml/hr 100% of the time (100% sensitivity), a key project goal indicating single test accuracy.

Real Time with Calibration to Zero-Flow

If a zero flow period can be established (e.g., the valve is set to its highest level or the shunt catheter is occluded for a short period of time) then results can be calibrated to reach a higher level of precision (as shown in FIG. 11B) which shows the CRT characteristic obtained in 288 experiments in 6 animals with zero-flow calibration). The error of the measurement decreases almost tenfold to 0.017 degrees for zero flow to 0.035 for 30 ml/h, SNR=15.77.

CRT sensor output correlated with flow rates with r²>0.9, meeting the program goal. This mode is particularly valuable in evaluating adjustable valves where zero flow can be established.

### CRT Tracks Changes in Flow Rates Over Time

The main goal of the testing program was to demonstrate that CRT ShuntCheck prototype concept can perform continuous testing, reliably identifying periods of shunt flow and non-flow. Bench and animal models were used, along with the CRT ShuntCheck prototype, to detect randomized periods of surrogate CSF fluid flowing at 0, 5, 10, 20 ml/h for durations of 5 minutes each induced by a syringe pump. In order to show how the CRT sensor follows the flow changes in time domain the graphs of FIGS. 12A and 12B are provided. The first graph (FIG. 12A) shows responses to a pulsing flow pattern of randomized flows within 0-10 ml/h range (flow rates are 0, 5, 7.5, 10 ml/h). Flow rates are changed every 5 minutes. The time domain response shows that the nature of the pattern is preserved in the CRT response. The response precisely follows each step-like change in the flow pattern. From this data, it can be concluded that the CRT ShuntCheck prototype presents a superior dynamic response compare to radionuclide and traditional ShuntCheck methods.

The dynamic changes of the flow rate are followed by the CRT signal. The phase shift or time delay between the signal and the flow rate is caused by physical delay in heat transfer. The time response of the CRT system is approximately 170 seconds, sufficiently quick to track natural fluctuations in CSF flow.

FIG. 12B shows that subtle changes in flow rate are integrated by the system, which is typical for a first order system. Since the CRT system is the first order system, it does not “overshoot”. The system always gradually approaches the steady state level.

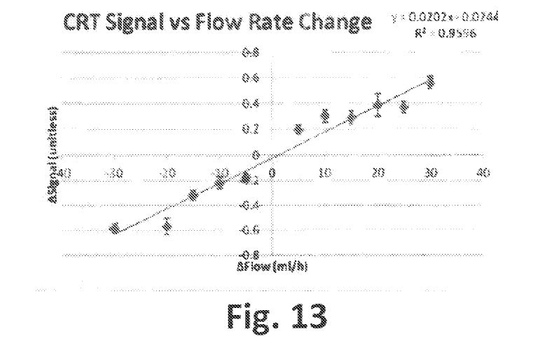

FIG. 13 demonstrates the sensitivity and accuracy of CRT in detecting changes in CSF flow (its most important diagnostic function). This graph summarizes all flow rate changes included in the bench and animal testing and shows that increases and decreases in flow are clearly detected and relatively well quantified (moderate increases in flow can be differentiated from significant changes—this is important for valve adjustment and for investigation of suspected over-drainage).

### CRT is Safe for Use Over Extended Time Periods

The sensor delivers very little cooling (skin temperature under the cooling device was 20° C.−the skin temperature target used in cosmetic laser surgery procedures to protect surrounding epidermis) on a very small surface area (4 mm×15 mm). This level of cooling caused no any observable effects on animal skin. The skin surface after 4 hour of cooling was pink and looked healthy. The cross section of the skin revealed no pathologies.

A literature search indicates that skin and tissue cooling is safe within a wide range of temperatures from 28° C. through 10.8° C. In accordance with one source, tissue cooling wasn't harmful even if applied for prolonged periods of time. A variety of cooling techniques have been implemented and are known for research and therapeutic purposes. Those techniques range from local ice pouches to global limb or body cooling. The most analogous study demonstrated that an aluminum probe cooled to −15° C. reduced skin temperature to 0° C. Skin freezing occurs at −2.2° C. of skin temperature and therefore requires a probe temperature of approximately −17° C. The probe temperature during the test reached a temperature averaging 20.7° C. (coldest 17.8° C.), yielding no skin damage.

The amount of heat per second removed by the probe from skin is less than 0.1 J (1 Amp×2V×5% efficiency) which is equivalent of elevating the temperature of 1 g of water by 0.0239 degrees C. This amount of cold is minute and easily compensated in the tissue volume by bio-heat generated by the human body, The surrounding skin stays warm even after hours of experimentation, The human body generates 0.01 J/sec per cubic centimeter of tissue. Thus only 10 cc of human tissue would be enough to completely negate the effect of the Peltier cooling.

In accordance with all of the test data and the CRT ShuntCheck prototype, the following conclusions have been reached:

1. CRT ShuntCheck demonstrates single test accuracy equal to current ShuntCheck.

2. CRT can track changes in CSF flow and can differentiate 0, 5, 10 and 20 ml/hr flow.

3 CRT is safe—Peltier cooling is precisely controlled by the device and the modest level of skin cooling is safe for extended test periods.

CRT ShuntCheck provides a noninvasive and safe method of CSF flow rate assessment. It can be utilized as a flow detector and as a flow change sensor (tracking changes in flow rate).

While the invention has been described in detail and with reference to specific examples thereof, it will be apparent to one skilled in the art that various changes and modifications can be made therein without departing from the spirit and scope thereof.

1. An apparatus for determining cerebrospinal fluid (CSF) flow rate in an implanted CSF shunt in real-time, said apparatus comprising:
a pad that is placed against the skin of a patient over the location of the CSF shunt, said pad comprising:
a Peltier sensor comprising:
a Peltier device that is operated continuously and which is displaced away from a first surface of said pad via a thermal resistor over the location of the shunt; and
a first set of temperature sensors associated with said Peltier device for detecting heat generated from the patient's skin and any CSF flow through the shunt; and
a sensor processing device that is electrically coupled to said pad for receiving temperature data from said first set of temperature sensors, said temperature data from said first set of temperature sensors being used by said sensor processing device to determine a real-time flow rate of the CSF through the CSF shunt.

2. The apparatus of claim 1 wherein one of said first temperature sensors is positioned between said Peltier device and a first surface of said thermal resistor and is used to control said Peltier device to a predetermined temperature.

3. The apparatus of claim 2 wherein another one of said first temperature sensors is positioned along a second surface of said pad which is positioned against patient's skin and wherein said another one of said first temperature sensors is also in thermal communication with a second surface of said thermal resistor;

4. The apparatus of claim 3 wherein said Peltier sensor further comprises a second set of temperature sensors arranged upstream and downstream of said Peltier device for detecting a temperature distribution along a path of the CSF shunt, said second set of temperature sensors generating a second set of temperature data that is used by said sensor processing device to define a zero flow baseline signal for calibrating said Peltier sensor.

5. The apparatus of claim 4 wherein said second set of temperature sensors comprises a first temperature sensor located upstream of said Peltier device and a second temperature sensor located downstream of said Peltier device when said pad is positioned over the location of the CSF shunt.

6. The apparatus of claim 5 wherein said second set of temperature sensors further comprises at least a third and a fourth temperature sensor located away from said Peltier device and away from the location of the CSF shunt, said third and fourth temperature sensors acting as control sensors for detecting skin temperature remote from the shunt.

7. The apparatus of claim 6 wherein said at least third and fourth temperatures sensors are located on opposite sides of said second temperature sensor.

8. The apparatus of claim 1 wherein said sensor processing device utilizes temperature data from said first set of temperature sensors to detect a temperature gradient between said first set of temperature sensors to form a Peltier signal, said Peltier signal corresponding to the CSF flow rate.

9. The apparatus of claim 1 wherein said Peltier device comprises a radiator and fan for heat dissipation.

10. The apparatus of claim 1 wherein each of said temperature sensors is a thermistor.

11. The apparatus of claim 1 wherein said pad is a flexible patch.

12. The apparatus of claim 1 wherein said predetermined temperature is maintained below the core temperature of the patient, thereby creating a temperature gradient such that any heat created by a CSF flow will be driven towards said Peltier device.

13. A method for determining cerebrospinal fluid (CSF) flow rate in an implanted CSF shunt in real-time, said method comprising:
applying a Peltier device, via a thermal resistor, against the skin of a patient over the location of the CSF shunt;
positioning a first temperature sensor between said Peltier device and said thermal resistor and positioning a second temperature sensor between said thermal resistor and the skin of the patient;
energizing said Peltier device on a continuous basis and using said first temperature sensor to control said Peltier device to maintain a predetermined temperature;
detecting a temperature gradient between said first and second temperature sensors from temperature data generated by said first and second temperature sensors; and
processing said temperature gradient to determine a Peltier signal that corresponds to a continuous real-time flow rate of said CSF through said CSF shunt.

14. The method of claim 13 wherein said step of positioning a first temperature sensor further comprises:
positioning a third temperature sensor upstream of said Peltier device over the location of the CSF shunt and positioning a fourth temperature sensor downstream of said Peltier device over the location of the CSF shunt;
positioning at least two additional temperature sensors, away from the location of the CSF shunt and said Peltier device; and
processing temperature data from said third, fourth and said at least two additional temperature sensors to define a zero flow baseline signal.

15. The method of claim 13 wherein said step of processing said temperature gradient further comprises: utilizing said zero flow baseline signal to calibrate said temperature gradient before using said temperature gradient to form said Peltier signal.

16. The method of claim 13 wherein said first and second temperature sensors are used to detect the heat generated from the patient's skin and any CSF flow over the location of the shunt.

17. The method of claim 14 wherein said step of positioning said at least two additional temperature sensors comprises aligning said at least two additional temperature sensors with said fourth temperature sensor.

18. The method of claim 17 wherein said at least two additional temperature sensors are located on opposite sides of said fourth temperature sensor.

19. The method of claim 13 wherein said step of energizing said Peltier device on a continuous basis comprises applying a radiator and to an exposed surface of said Peltier device for heat dissipation

20. The method of claim 13 wherein said of energizing said Peltier device on a continuous basis comprises energizing a fan on an exposed side of said Peltier device for heat dissipation.

21. The method of claim 13 wherein step of step of applying a Peltier device to the skin of the patient comprises coupling said Peltier device, via said thermal resistor, to a flexible patch that is applied directly to the skin of the patient.

22. The method of claim 13 wherein said step of energizing said Peltier device comprises maintaining said predetermined temperature that is below the core temperature of the patient, thereby creating a temperature gradient such that any heat created by a CSF flow will be driven towards said Peltier device.

23. An apparatus for determining cerebrospinal fluid (CSF) flow rate in an implanted CSF shunt in real-time, said apparatus comprising:
a pad that is placed against the skin of a patient over the location of the CSF shunt, said pad comprising:
a Peltier sensor comprising:
a Peltier device that is operated continuously with a first set of temperature sensors associated with said Peltier device for detecting heat generated from the patient's skin and any CSF flow over the location of the shunt;
a second set of temperature sensors arranged upstream and downstream of said Peltier device for detecting a temperature distribution along a path of the CSF shunt; and
a sensor processing device that is electrically coupled to said pad for receiving temperature data from said first set of temperature sensors and from said second set of temperature sensors, said temperature data from said second set of temperature sensors being used by said sensor processing device to define a zero flow baseline signal for calibrating said Peltier sensor and wherein said sensor processing device further uses said temperature data from said first set of temperature sensors in conjunction with said zero flow baseline signal to determine a continuous real-time flow rate of the CSF through the CSF shunt.

24. The apparatus of claim 23 wherein said Peltier device is displaced away from a first surface of said pad via a thermal resistor and wherein one of said first set of temperature sensors is positioned between said Peltier device and a first surface of said thermal resistor, said one of said first set of temperature sensors being used to control said Peltier device to a predetermined temperature.

25. The apparatus of claim 24 wherein another one of said first set of temperature sensors is positioned along a second surface of said pad that is positioned against the patient's skin, said another one of said first set of temperature sensors being used to detect the heat generated from the patient's skin and any CSF flow over the location of the shunt.

26. The apparatus of claim 25 wherein said another one of said first set of temperature sensors is also positioned to be in thermal communication with a second surface of said thermal resistor.

27. The apparatus of claim 23 wherein said second set of temperature sensors comprises a first temperature sensor located upstream of said Peltier device and a second temperature sensor located downstream of said Peltier device when said pad is positioned over the location of the CSF shunt.

28. The apparatus of claim 27 wherein said second set of temperature sensors further comprises at least a third and a fourth temperature sensor located away from said Peltier device and away from the location of the CSF shunt, said third and fourth temperature sensors acting as control sensors for detecting skin temperature remote from the shunt.

29. The apparatus of claim 27 wherein said second set of temperature sensors further comprises at least a third and a fourth temperature sensor located away from said Peltier device and away from the location of the CSF shunt, said third and fourth temperature sensors being transversely aligned with said second temperature sensor located downstream of said Peltier device, and acting as control sensors for detecting skin temperature remote from the shunt.

30. The apparatus of claim 29 wherein said at least third and fourth temperatures sensors are located on opposite sides of said second temperature sensor.

31. The apparatus of claim 25 wherein said sensor processing device utilizes temperature data from said first set of temperature sensors to detect a temperature gradient between said first set of temperature sensors to form a Peltier signal, said Peltier signal corresponding to the CSF flow rate.

32. The apparatus of claim 23 wherein said Peltier device comprises a radiator and fan for heat dissipation.

33. The apparatus of claim 23 wherein each of said temperature sensors is a thermistor.

34. The apparatus of claim 23 wherein said pad is a flexible patch.

35. The apparatus of claim 24 wherein said predetermined temperature is maintained below the core temperature of the patient, thereby creating a temperature gradient such that any heat created by a CSF flow will be driven towards said Peltier device.
